(19) United States
(12) Patent Application Publication
(10) Pub. No.: US 2008/0041370 A1
(43) Pub. Date: Feb. 21, 2008

(54) AEROSOL FORMULATION FOR THE INHALATION OF BETA AGONISTS

(76) Inventors: Kirsten RADAU, Ingelheim (DE); Michael AVEN, Mainz (DE); Rainer WEITZEL, Schwabenheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/839,817

(22) Filed: Aug. 16, 2007

(30) Foreign Application Priority Data
Aug. 18, 2006 (EP) 06119129.2
Jan. 25, 2007 (EP) 07101128.2

(51) Int. Cl. A61M 11/02; A61K 31/535; A61K 31/56; A61K 31/58; A61M 11/00

(52) U.S. Cl. 128/200.23; 128/200.14; 514/178; 514/230.5; 514/514

(57) ABSTRACT

The present invention relates to a propellant-free aerosol formulation which contains one or more compounds of general formula 1,

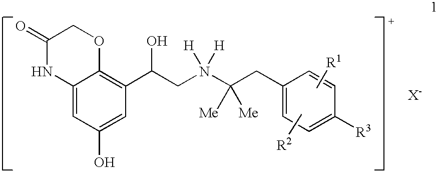

wherein the groups R¹, R², R³ and X⁻ may have the meanings indicated in the claims and in the specification, and one other active substance 2, for inhalation.

This application claims priority benefit from EP 06 119 129.2, filed Aug. 18, 2006; and EP 07 101 128.2, filed Jan. 25, 2007, all of which are incorporated herein in their entirety.

The present invention relates to a propellant-free aerosol formulation which contains one or more compounds of general formula 1,

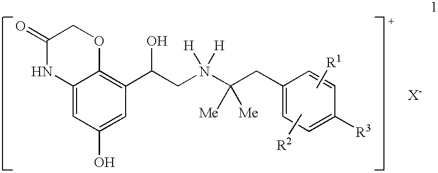

wherein the groups R¹, R², R³ and X⁻ may have the meanings indicated in the claims and in the specification, and one other active substance 2, for inhalation.

### DETAILED DESCRIPTION OF THE INVENTION

The medicament formulations according to the invention are propellant-free medicament formulations, containing as active substance one or more compounds of general formula 1

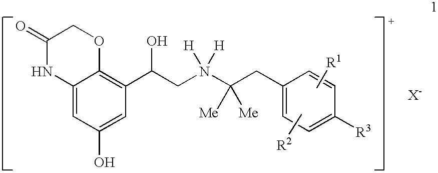

wherein
R¹ denotes hydrogen, C₁₋₄-alkyl, O—C₁₋₄-alkyl or halogen;
R² denotes hydrogen, C₁₋₄-alkyl, O—C₁₋₄-alkyl or halogen;
R³ denotes hydrogen, C₁₋₄-alkyl, O—C₁₋₄-alkyl, halogen, OH, —O—C₁₋₄-alkylene-COOH or O—C₁₋₄-alkylene-COO—C₁₋₄-alkyl,
X— denotes a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   optionally in the form of the tautomers, enantiomers, mixtures of the enantiomers, racemates, solvates or hydrates thereof; an active substance 2 selected from among budesonide, beclomethasone, fluticasone, ciclesonide or a metabolite thereof, optionally in the form of the tautomers, enantiomers, mixtures of the enantiomers, racemates, solvates or hydrates thereof; at least one pharmacologically acceptable acid or a pharmacologically acceptable buffer system, optionally further pharmacologically acceptable excipients, as well as ethanol or a mixture of water and ethanol as the solvent.

Preferred medicament formulations are those that contain the above-mentioned active substance 2 and compounds of general formula 1, wherein
R¹ denotes hydrogen, methyl, ethyl, fluorine or chlorine;
R² denotes hydrogen, methyl, ethyl, fluorine or chlorine;
R³ denotes hydrogen, methyl, ethyl, propyl, OH, methoxy, ethoxy, fluorine, chlorine, bromine, O—CH₂—COOH, O—CH₂—COOmethyl or O—CH₂—COOethyl, —O—CH₂—CH₂COOH, O—CH₂—CH₂COOmethyl or O—CH₂—CH₂COOethyl, —O—CH₂—CH₂—CH₂COOH, O—CH₂—CH₂—CH₂COOmethyl or —O—CH₂—CH₂—CH₂COOethyl;
X— denotes a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   optionally in the form of the tautomers, enantiomers, mixtures of the enantiomers, racemates, solvates or hydrates thereof.

Preferred medicament formulations are those that contain the above-mentioned active substance 2 and compounds of general formula 1, wherein
R¹ denotes hydrogen or methyl, preferably hydrogen;
R² denotes hydrogen or methyl, preferably hydrogen;
R³ denotes methyl, OH, methoxy, fluorine, chlorine, bromine, O—CH₂—COOH or —O—CH₂—COOethyl;
X— denotes a mono- or polysubstituted negatively charged anion selected from among chloride, bromide, sulphate, methanesulphonate, maleate, acetate, benzoate, citrate, salicylate, trifluoroacetate, fumarate, tartrate and succinate;
   optionally in the form of the tautomers, enantiomers, mixtures of the enantiomers, racemates, solvates or hydrates thereof.

Also preferred are medicament formulations that contain the above-mentioned active substance 2 and compounds of general formula 1, wherein
R³ denotes methoxy, ethoxy, fluorine, chlorine, bromine, O—CH₂—COOH, —O—CH₂—COOmethyl or O—CH₂—COOethyl;
   and R¹, R² and X⁻ may have the meanings given above, optionally in the form of the tautomers, enantiomers, mixtures of the enantiomers, racemates, solvates or hydrates thereof.

Also preferred are medicament formulations that contain the above-mentioned active substance 2 and compounds of general formula 1, wherein
R¹ denotes hydrogen;
R² denotes hydrogen;
R³ denotes OH, fluorine, chlorine, methoxy, ethoxy, —O—CH₂—COOH, preferably OH, fluorine, chlorine, ethoxy or methoxy,
   and X⁻ may have one of the meanings given above, optionally in the form of the tautomers, enantiomers, mixtures of the enantiomers, racemates, solvates or hydrates thereof.

Also preferred are medicament formulations that contain the above-mentioned active substance 2 and the compounds of general formula 1 which are selected from among:
6-hydroxy-8-{1-hydroxy-2-[2-(4-methoxy-phenyl)-1,1-dimethyl-ethylamino]-ethyl}-4H-benzo[1,4]oxazin-3-one;
6-hydroxy-8-{1-hydroxy-2-[2-(ethyl 4-phenoxy-acetate)-1,1-dimethyl-ethylamino]-ethyl}-4H-benzo[1,4]oxazin-3-one;
6-hydroxy-8-{1-hydroxy-2-[2-(4-phenoxy-acetic acid)-1,1-dimethyl-ethylamino]-ethyl}-4H-benzo[1,4]oxazin-3-one;
8-{2-[1,1-dimethyl-2-(2.4.6-trimethylphenyl)-ethylamino]-1-hydroxy-ethyl}-6-hydroxy-4H-benzo[1,4]oxazin-3-one;
6-hydroxy-8-{1-hydroxy-2-[2-(4-hydroxy-phenyl)-1,1-dimethyl-ethylamino]-ethyl}-4H-benzo[1,4]oxazin-3-one;
6-hydroxy-8-{1-hydroxy-2-[2-(4-isopropyl-phenyl)-1,1-dimethyl-ethylamino]-ethyl}-4H-benzo[1,4]oxazin-3-one;
8-{2-[2-(4-ethyl-phenyl)-1,1-dimethyl-ethylamino]-1-hydroxy-ethyl}-6-hydroxy-4H-benzo[1,4]oxazin-3-one;
8-{2-[2-(4-fluoro-3-methyl-phenyl)-1,1-dimethyl-ethylamino]-1-hydroxy-ethyl}-6-hydroxy-4H-benzo[1,4]oxazin-3-one;
8-{2-[2-(4-fluoro-2-methyl-phenyl)-1,1-dimethyl-ethylamino]-1-hydroxy-ethyl}-6-hydroxy-4H-benzo[1,4]oxazin-3-one;
8-{2-[2-(2,4-difluoro-phenyl)-1,1-dimethyl-ethylamino]-1-hydroxy-ethyl}-6-hydroxy-4H-benzo[1,4]oxazin-3-one;
8-{2-[2-(3,5-difluoro-phenyl)-1,1-dimethyl-ethylamino]-1-hydroxy-ethyl}-6-hydroxy-4H-benzo[1,4]oxazin-3-one;
8-{2-[2-(4-ethoxy-phenyl)-1,1-dimethyl-ethylamino]-1-hydroxy-ethyl}-6-hydroxy-4H-benzo[1,4]oxazin-3-one;
8-{2-[2-(3,5-dimethyl-phenyl)-1,1-dimethyl-ethylamino]-1-hydroxy-ethyl}-6-hydroxy-4H-benzo[1,4]oxazin-3-one;
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid;
8-{2-[2-(3,4-difluoro-phenyl)-1,1-dimethyl-ethylamino]-1-hydroxy-ethyl}-6-hydroxy-4H-benzo[1,4]oxazin-3-one;
8-{2-[2-(2-chloro-4-fluoro-phenyl)-1,1-dimethyl-ethylamino]-1-hydroxy-ethyl}-6-hydroxy-4H-benzo[1,4]oxazin-3-one;
8-{2-[2-(4-chloro-phenyl)-1,1-dimethyl-ethylamino]-1-hydroxy-ethyl}-6-hydroxy-4H-benzo[1,4]oxazin-3-one;
8-{2-[2-(4-bromo-phenyl)-1,1-dimethyl-ethylamino]-1-hydroxy-ethyl}-6-hydroxy-4H-benzo[1,4]oxazin-3-one;
8-{2-[2-(4-fluoro-phenyl)-1,1-dimethyl-ethylamino]-1-hydroxy-ethyl}-6-hydroxy-4H-benzo[1,4]oxazin-3-one;
8-{2-[2-(4-fluoro-3-methoxy-phenyl)-1,1-dimethyl-ethylamino]-1-hydroxy-ethyl}-6-hydroxy-4H-benzo[1,4]oxazin-3-one;
8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;
8-{2-[2-(4-chloro-2-methyl-phenyl)-1,1-dimethyl-ethylamino]-1-hydroxy-ethyl}-6-hydroxy-4H-benzo[1,4]oxazin-3-one;
8-{2-[2-(4-chloro-3-fluoro-phenyl)-1,1-dimethyl-ethylamino]-1-hydroxy-ethyl}-6-hydroxy-4H-benzo[1,4]oxazin-3-one;
8-{2-[2-(4-chloro-2-fluoro-phenyl)-1,1-dimethyl-ethylamino]-1-hydroxy-ethyl}-6-hydroxy-4H-benzo[1,4]oxazin-3-one;
8-{2-[2-(3-chloro-4-fluoro-phenyl)-1,1-dimethyl-ethylamino]-1-hydroxy-ethyl}-6-hydroxy-4H-benzo[1,4]oxazin-3-one;
8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;
8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;
8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;
8-{2-[2-(3,5-dichloro-phenyl)-1,1-dimethyl-ethylamino]-1-hydroxy-ethyl}-6-hydroxy-4H-benzo[1,4]oxazin-3-one;
8-{2-[2-(4-chloro-3-methyl-phenyl)-1,1-dimethyl-ethylamino]-1-hydroxy-ethyl}-6-hydroxy-4H-benzo[1,4]oxazin-3-one;
8-{2-[2-(3,4,5-trifluoro-phenyl)-1,1-dimethyl-ethylamino]-1-hydroxy-ethyl}-6-hydroxy-4H-benzo[1,4]oxazin-3-one;
8-{2-[2-(3-methyl-phenyl)-1,1-dimethyl-ethylamino]-1-hydroxy-ethyl}-6-hydroxy-4H-benzo[1,4]oxazin-3-one and
8-{2-[2-(3,4-dichloro-phenyl)-1,1-dimethyl-ethylamino]-1-hydroxy-ethyl}-6-hydroxy-4H-benzo[1,4]oxazin-3-one,
   in each case in the form of an acid addition salts with an acid HX, wherein X⁻ may have one of the meanings given above, and optionally in the form of the tautomers, enantiomers, mixtures of the enantiomers, racemates, solvates or hydrates thereof.

In the medicament combinations according to the invention the active substance 2 is selected from among the group of steroids comprising budesonide, beclomethasone, fluticasone, ciclesonide or a metabolite thereof. The above-mentioned steroids may optionally have chiral carbon centres. In this case the medicament combinations according to the invention may contain the steroids in the form of the enantiomers, mixtures of enantiomers or racemates thereof, while steroids with high enantiomeric purity are preferably used.

### Terms and Definitions Used

By the term “C₁₋₄-alkyl” (including those which are part of other groups) are meant branched and unbranched alkyl groups with 1 to 4 carbon atoms. Examples include: methyl, ethyl, n-propyl, i-propyl, n-butyl, i-butyl, sec-butyl or tert-butyl. The following abbreviations may optionally also be used for the above-mentioned groups: Me, Et, n-Pr, i-Pr, n-Bu, i-Bu, t-Bu, etc. Unless stated otherwise, the definitions propyl and butyl include all the possible isomeric forms of the groups in question. Thus, for example, propyl includes n-propyl and i-propyl, butyl includes i-butyl, sec-butyl and tert-butyl etc.

By the term “C₁₋₄-alkylene” (including those which are part of other groups) are meant branched and unbranched alkylene groups with 1 to 4 carbon atoms. Examples include: methylene, ethylene, propylene, 1-methylethylene, butylene, 1-methylpropylene, 1,1-dimethylethylene or 1,2-dimethylethylene. Unless stated otherwise, the definitions propylene and butylene include all the possible isomeric forms of the groups in question with the same number of carbons. Thus, for example, propylene also includes 1-methylethylene and butylene includes 1-methylpropylene, 1,1-dimethylethylene, 1,2-dimethylethylene.

“Halogen” within the scope of the present invention represents fluorine, chlorine, bromine or iodine. Unless stated to the contrary, fluorine, chlorine and bromine are regarded as preferred halogens.

By acid addition salts with pharmacologically acceptable acids are meant for example salts selected from among hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate. Of the above-mentioned acid addition salts the salts of hydrochloric acid, methanesulphonic acid, benzoic acid and acetic acid are particularly preferred according to the invention.

By compounds with high enantiomeric purity are meant those compounds that may consist of two or more enantiomers, in which one enantiomer is present in excess, the excess is preferably more than 90%, particularly preferably more than 95%, and especially more than 98% of the total mass.

By metabolites of the steroids are meant, for the purposes of the invention, steroids that result from the metabolism or that are reacted in the metabolism. Thus, it may be that the pharmaceutically active steroid actually corresponds to a metabolite of the steroid used. If the metabolites are pharmaceutically stable they may also be used directly. Thus, for example, des-ciclesonide when administered into the lung is a pharmaceutically active metabolite of ciclesonide (D. Ukena, Pneumologie 2005; 59; 689-695).

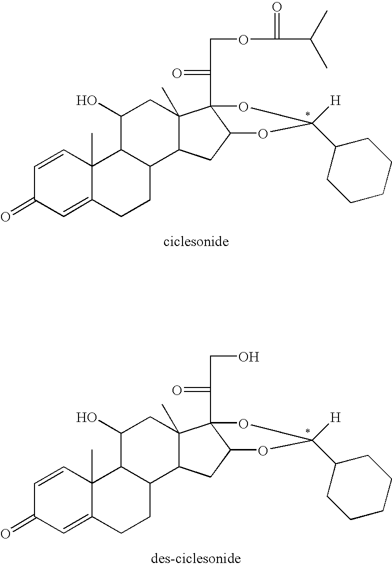

The compounds according to the invention may be prepared analogously to the methods already known in the art. Suitable methods of preparation are known for example from U.S. Pat. No. 4,460,581, the contents of which are incorporated herein by reference.

The compounds of formula 1 may optionally be present in the medicament formulations according to the invention in the form of their tautomers. The term tautomerism denotes the occurrence of isomeric compounds which are formed by displacing σ- or π-bonds and which may be present in equilibrium. Examples of possible tautomeric forms of the compounds of formula 1 are

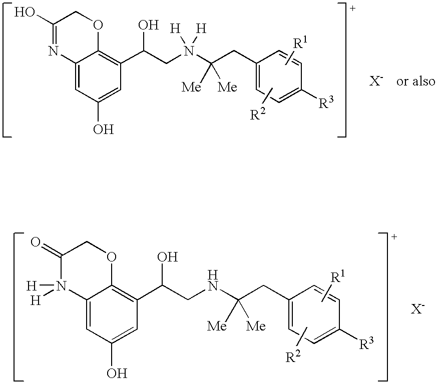

In another aspect the present invention relates to medicament formulations that contain the above-mentioned compounds of formula 1 in the form of the individual optical isomers, mixtures of the individual enantiomers or racemates. Particularly preferred are medicament formulations which contain the above-mentioned compounds of formula 1 in the form of the compounds with high enantiomeric purity, while the R-enantiomers of the compounds of formula 1 are of exceptional importance according to the invention. These R-enantiomers may be represented by general formula R-1

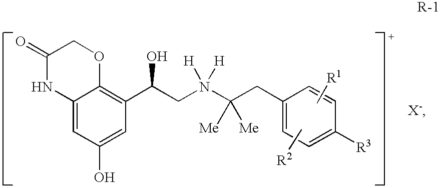

wherein the groups R¹, R², R³ and X⁻ may have the meanings given above.

Within the scope of the present invention it is particularly preferable to use those compounds of formula 1 wherein X⁻ is selected from among chloride, maleate, salicylate, fumarate or succinate, optionally in the form of the hydrates and solvates thereof. Particularly preferred within the scope of the present invention are those formulations that contain the compound of formula 1 wherein X⁻ denotes chloride.

References to the compound of formula 1 always include within the scope of the present invention all the possible amorphous and crystalline modifications of this compound. References to the compound of formula 1 also include within the scope of the present invention all the possible solvates and hydrates which may be formed from this compound. Any reference made to the compound 1′ within the scope of the present invention is to be regarded as a reference to the pharmacologically active free base of the following formula contained in the salts 1:

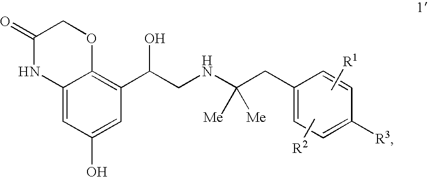

wherein the groups R¹, R² and R³ may have the meanings given above.

In another aspect the present invention relates to medicament formulations containing an active substance 2 and a free base of formula 1′ wherein the groups R¹, R² and R³ may have the meanings given above, optionally in the form of the tautomers, enantiomers, mixtures of the enantiomers, racemates, solvates or hydrates thereof, at least one pharmacologically acceptable acid, optionally further pharmacologically acceptable excipients, as well as water, ethanol or a mixture of water and ethanol as solvent.

In another aspect the present invention relates to the use of the medicament formulations according to the invention for preparing a pharmaceutical composition for the treatment of respiratory complaints, which are selected from among obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the compounds are used as described above to prepare a pharmaceutical composition for the treatment of obstructive pulmonary diseases selected from among bronchial asthma, paediatric asthma, severe asthma, acute asthma attacks, chronic bronchitis and chronic obstructive pulmonary diseases (COPD), while it is particularly preferable according to the invention to use them for preparing a pharmaceutical composition for the treatment of bronchial asthma or COPD.

It is also preferable to use the medicament formulations according to the invention to prepare a medicament for the treatment of pulmonary emphysema which has its origins in COPD (chronic obstructive pulmonary disease) or α1-proteinase inhibitor deficiency.

It is also preferable to use the medicament formulations according to the invention to prepare a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchioalveolar carcinoma and lymphomas.

It is also preferable to use the medicament formulations according to the invention to prepare a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament formulations according to the invention to prepare a pharmaceutical composition for the treatment of cystic fibrosis or mucoviscidosis.

It is also preferable to use the medicament formulations according to the invention to prepare a pharmaceutical composition for the treatment of bronchitis, such as for example bronchitis caused by bacterial or viral infection, allergic bronchitis and toxic bronchitis.

It is also preferable to use the medicament formulations according to the invention to prepare a pharmaceutical composition for the treatment of bronchiectasis.

It is also preferable to use the medicament formulations according to the invention to prepare a pharmaceutical composition for the treatment of ARDS (adult respiratory distress syndrome).

It is also preferable to use the medicament formulations according to the invention to prepare a pharmaceutical composition for the treatment of pulmonary oedema, for example toxic pulmonary oedema after aspiration or inhalation of toxic substances and foreign substances.

Particularly preferably, the present invention relates to the use of the medicament formulations according to the invention for preparing a pharmaceutical composition for the treatment of asthma or COPD. Also of particular importance is the above-mentioned use for preparing a pharmaceutical composition for once-a-day treatment of inflammatory and obstructive respiratory complaints, particularly for the once-a-day treatment of asthma or COPD.

Moreover, according to a further aspect, the present invention relates to the use of the medicament formulations according to the invention for preparing a pharmaceutical composition for stimulating stem cell mobilisation.

The present invention also relates to a process for the treatment of the above-mentioned ailments, characterised in that one or more of the above-mentioned medicament formulations according to the invention are administered in therapeutically effective amounts. It is particularly desirable to prepare an active substance formulation which can be used therapeutically by administration once a day (single dose). The use of a drug once a day has the advantage that the patient can become accustomed relatively quickly to regularly taking the drug at certain times of the day.

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of the active substances in the lung it makes sense to use a liquid formulation without propellant gases administered using suitable inhalers. A formulation of this kind may be inhaled both by oral route and by nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 25 microlitres of active substance solution can be nebulised preferably in one puff or two puffs to form an aerosol having an average particle size (or particle diameter) of less than 20 microns, preferably less than 10 microns, so that the inhalable part of the aerosol already corresponds to the therapeutically effective quantity. An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in Patent Application WO 94/07607 or in Patent Application WO 99/16530. Reference is expressly made here to both these publications.

The aim of the invention is to provide an aqueous, ethanolic or aqueous-ethanolic formulation of the compound of formula 1 which meets the high standards required to ensure optimum nebulisation of a solution using the inhalers mentioned above. The active substance formulations according to the invention must be of sufficiently high pharmaceutical quality, i.e. they should be pharmaceutically stable over a storage time of some years, preferably at least twelve months, more preferably eighteen months. These propellant-free formulations of solutions must also be capable of being nebulised by means of an inhaler under pressure, while the composition delivered in the aerosol produced is within a specified range.

According to the invention the formulation preferably contains the active substance 2 and only one compound of formula 1. However, the formulation may also contain a mixture of different salts of formula 1. If the medicament formulations according to the invention contain different salts of formula 1, the preferred formulations according to the invention are those wherein the various salts are different salts of the same free base of formula 1′.

The concentration of the compound of formula 1 based on the amount of pharmacologically active free base 1′ in the medicament formulation according to the invention is about 0.1 to 1000 mg pro 100 ml, preferably about 0.5 to 500 mg per 100 ml, particularly preferably 1 to 250 mg per 100 ml according to the invention. Particularly preferably 100 ml of the formulations according to the invention contain about 2 to about 100 mg of 1′.

The concentration of the compound of formula 2 in the medicament formulation according to the invention is about 10 to 6000 mg per 100 ml, preferably 10 to 5000 mg per 100 ml, preferably 50 to 5000 mg per 100 ml, preferably 50 to 3000 mg per 100 ml, particularly preferably 75 to 3500 mg per 100 ml, particularly preferably 75 to 2500 mg per 100 ml. Particularly preferably, 100 ml of the formulations according to the invention contain about 100 to about 2500 mg.

The medicament formulations according to the invention contain as solvent pure ethanol or mixtures of ethanol and water. If ethanol-water mixtures are used, the percentage amount of ethanol by volume in these mixtures is preferably in the range between 30 and 98% ethanol, particularly preferably in the range from 40 to 97% ethanol. Most particularly preferred medicament formulations for the purposes of the present invention contain as solvent pure ethanol or ethanol-water mixtures containing between 50 and 96%, particularly preferably between 67 and 95%, particularly between 67 and 93% ethanol. Besides ethanol and water it is also possible to use other co-solvents and solubilisers such as e.g. benzylalcohol, γ-butyrolactone or diethyleneglycol monoethylether. According to the invention, however, it is preferable if no additional solvent is used.

If the compounds 1 and 2 are dissolved in ethanol or in mixtures of ethanol and water, the pH of the formulation according to the invention is preferably in the range from 2.0 and 6.5, preferably between 2.5 and 5.5, particularly preferably between about 3.0 and 5.0, particularly between 2.8 and 4.8, according to the invention.

In another variant of the invention the pH of the formulation according to the invention is preferably in the range from 3.0 to 6.5, preferably between 3.5 and 6.5, particularly preferably between about 4.0 and 6.0.

The pH is adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid. Examples of particularly suitable organic acids are selected from the group consisting of ascorbic acid, citric acid, malic acid, tartaric acid, maleic acid, succinic acid, fumaric acid, acetic acid, formic acid, propionic acid, sorbic acid, benzoic acid, methanesulphonic acid and benzenesulphonic acid. Preferred inorganic acids are hydrochloric acid, phosphoric acid and sulphuric acid, of which hydrochloric acid and phosphoric acid are particularly important according to the invention. Of the organic acids, ascorbic acid, fumaric acid, methanesulphonic acid and citric acid are preferred, of which citric acid is particularly preferred according to the invention. If desired, mixtures of the abovementioned acids may also be used, particularly in the case of acids which have other properties in addition to their acidifying properties, e.g. those which act as flavourings or antioxidants, such as for example citric acid or ascorbic acid. If desired, pharmacologically acceptable bases may also be used to titrate the pH precisely. Suitable bases include for example alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the abovementioned acid.

Additionally, the pH may also be adjusted using a pharmacologically acceptable buffer system. For this, pharmacologically acceptable inorganic or organic buffer systems may be used. Examples of preferred buffer systems are selected from among citrate buffer, acetate buffer and phosphate buffer. Particularly preferred is the phosphate buffer.

The formulations according to the invention may contain complexing agents as further pharmacologically acceptable excipients. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA, as complexing agent. Preferably, disodium edetate is used, optionally in the form of its hydrates, more preferably in the form of its dihydrate. Moreover, EDTA may be present in the ethanol-containing solution in the form of its ethyl ester, and this may be in the form of the mono-, di-, tri- or tetraethyl ester or mixtures thereof.

If complexing agents are used within the scope of the formulations according to the invention, their content is preferably in the range from 0.1 to 50 mg per 100 ml, particularly preferably in the range from 0.25 to 25 mg per 100 ml of the formulation according to the invention. Preferably, the formulations according to the invention contain a complexing agent in an amount of about 0.3 to 15 mg per 100 ml, particularly preferably from about 0.4 to 8 mg per 100 ml.

The remarks made concerning disodium edetate also apply analogously to other possible additives which are comparable to EDTA or the salts thereof, which have complexing properties and can be used instead of them, such as for example nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, propylgallate and both natural and synthetic phenolic antioxidants. The natural phenolic antioxidants include for example vitamin A, tocopherols such as vitamin E, and similar vitamins or provitamins occurring in the human body. The natural antioxidants also include flavonoids occurring in plant organisms, such as e.g. naringenin and resveratrol. The synthetic antioxidants include e.g. BHA (butylhydroxyanisol), BHT (butylhydroxytoluene), TBHQ (tert-butylhydroxyquinone), tris(2,4-di-tert-butylphenyl)phosphite and tetrakis[methylene(3,5-di-tert-butylhydroxyhydrocinnamate)]methane. BHT or tocopherols are preferred, while BHT is most preferred.

If antioxidants are used within the scope of the formulations according to the invention, their content is preferably in the range from 0.1 to 200 mg per 100 ml.

Preservatives can be added to protect the formulation from contamination with pathogenic bacteria. Suitable preservatives are those known from the prior art, particularly benzalkonium chloride or benzoic acid or benzoates such as sodium benzoate in the concentrations known from the prior art. Preferably, benzalkonium chloride is added to the formulation according to the invention. The amount of benzalkonium chloride added is between 1 mg and 50 mg per 100 ml of formulation, preferably about 2 to 15 mg per 100 ml, particularly preferably about 3 to 12 mg per 100 ml, particularly preferably about 4 to 10 mg per 100 ml of the formulation according to the invention. Benzalkonium chloride may also be used according to the invention in admixture with other preservatives.

In the case of ethanol/water mixtures of 50 to 90% V/V there is no need for any additional preservative, as this property is already present in the solvent mixture.

Preferred formulations contain only an antioxidant, a complexing agent and the acid needed to adjust the pH, or a buffer system, besides the solvent water and ethanol, the compounds of formula 1 and active substance 2. Particularly preferred formulations contain only BHT, EDTA and the acid needed to adjust the pH, or a buffer system, besides the solvents water and ethanol, the compounds of formula 1 and the active substance 2.

### Nebulisers

The nebulisation of pharmaceuticals dissolved or suspended in water may be carried out using compressed air or ultrasound. The resulting particle spectrum is superior to propellant gas and powder aerosols in its delivery to the lungs. This method of inhalation is suitable for cases of severe asthma and because of the simple inhalation technique it is also suitable for children and patients who have problems coordinating their breathing. There are both stationary devices and small devices for use when travelling. These are naturally always larger than MDI's and DPI's. The pharmaceutical preparations that can be used are limited to microbiologically safe, aqueous, isotonic and pH-neutral solutions or suspension.

Jet nebulisers—For a long time, simple devices have been used for distributing solutions, in which a powerful air current is passed through the opening of a capillary tube through which the solution is sucked (the perfume atomiser principle). In hand-held atomisers made of glass (nebulisers) the air current is generated by compressing a rubber ball or by pumping (pump atomiser). More recent stationary devices for aerosol therapy are nebulisers operating by compressed air which are able to generate an amount of over 50% in the optimum size range (1-5 μm). Compressed air is accelerated through a nozzle and carries the medicament solution through capillaries (Bernoulli effect), during which time the solution is dispersed. An impact plate located behind the nozzle additionally serves to break up the solution. Special blocking means ensure that only the smallest particles escape, while the larger particles flow back into the reservoir and can be nebulised again. During inhalation considerable evaporation takes place, which leads to a cool aerosol and concentration of the active substance solution, as a result of the coldness of evaporation.

Ultrasound nebulisers—A piezoelectric crystal is excited, by high-frequency alternating current, to produce vibrations which are transmitted through a transfer medium to the active substance solution and from it release very fine droplets of liquid but at the same time heat the liquid.

The medicament formulations according to the invention are preferably used in an inhaler of the type described hereinbefore. to produce the propellant-free aerosols according to the invention. At this point we should once again expressly mention the patent documents described hereinbefore, to which reference is hereby made. As described at the beginning, a further developed embodiment of the preferred inhaler is disclosed in WO 97/12687 (cf. in particular FIGS. 6a and 6b and the associated passages of description).

This nebuliser (Respimat®) can advantageously be used to produce the inhalable aerosols according to the invention. Because of its cylindrical shape and handy size of less than 9 to 15 cm long and 2 to 4 cm wide, the device can be carried anywhere by the patient. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 3—and the associated parts of the description of the above-mentioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance solution. Volumes of 10 to 50 microlitres are preferred, volumes of 10 to 20 microlitres are more preferable, whilst a volume of 10 to 17.5 microlitres per actuation is particularly preferred.

The valve body is preferably mounted at the end of the hollow piston which faces the nozzle body.

The nozzle in the nozzle body is preferably microstructured, i.e. manufactured by micro-engineering. Microstructured nozzle bodies are disclosed for example in Patent Application WO 99/16530; reference is hereby made to the contents thereof, especially FIG. 1 and the associated description. The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns. If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°. The nozzle openings are preferably arranged at a spacing of 10 to 200 microns, more preferably at a spacing of 10 to 100 microns, still more preferably 30 to 70 microns. A spacing of 50 microns is most preferred. The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably up to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the tensioning of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray.

Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6 a/b of WO 97/12687 show the nebuliser (Respimat®) with which the aqueous aerosol preparations according to the invention can advantageously be inhaled. FIG. 6 a shows a longitudinal section through the atomiser with the spring under tension, FIG. 6 b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the method described above (Respimat®), the mass expelled, in at least 97%, preferably at least 98% of all the actuations of the inhaler (puff or puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

The formulation according to the invention can also be nebulised using inhalers other than those described above, for example jet-stream inhalers or liquid drop inhalers.

The present invention also relates to an inhalation kit consisting of one of the pharmaceutical preparations according to the invention described above and an inhaler suitable for nebulising this pharmaceutical preparation. The present invention preferably relates to an inhalation kit consisting of one of the pharmaceutical preparations according to the invention described above and the Respimat® inhaler described above.

If the formulation is to be administered nasally using the Respimat® device described above, this atomiser can be provided with an attachment on the mouthpiece which is designed in the manner of a cylindrical pyramid, i.e. a pyramid with a round or oval cross-section or a tapering, round or oval cylinder. This attachment is hollow on the inside and has two openings. One of the openings may be fitted over the mouthpiece and the other opening at the pointed end can be inserted in a nostril.

Thus, this attachment is preferably in the form of the spout of a conventional nasal spray. The attachment may be constructed so as to be detachably or non-detachably connected to the mouthpiece. An attachment of this kind may also replace the mouthpiece.

The inhalable solution is contained in a suitable gas- and fluid-tight container, the capacity of which is adapted to the intended use, and thus container collapses plastically and irreversibly in a predetermined manner under slightly reduced pressure and can be emptied almost totally.

This problem is solved according to the invention by a container for a medicinal liquid which is gas- and fluid-tight and which is characterised by
a film bag sealed at both ends and which is deformable and collapses as a result of the external pressure when there is a pressure difference between the interior of the container and its environment of less than 300 hPa (300 mbar),
and an inherently rigid flange which is tightly connected to the film bag and which is constructed as a detachable connecting element for fitting the container onto a removal nozzle,
and at least one weld seam by which the film bag is closed off at least at one end and which extends substantially at right angles to the axis of the bag,
and a sealing point in the inherently rigid flange,
and a removal point for the liquid in the region of the inherently rigid flange.

In another embodiment the collapsible film bag may be deformed and collapsed by the external pressure at a differential pressure of below 150 hPa (150 mbar) or preferably below 80 hPa (80 mbar).

The film bag may be closed off by a weld seam at both ends. In this case the inherently rigid flange is welded tightly to the side of the film bag, preferably close to one end of the film bag. However, the film bag may also be tightly sealed off at one end by a weld seam and at the other end by the inherently rigid flange. In this case, one end of the film bag is welded to the inherently rigid flange, preferably at its periphery. The inherently rigid flange may take various forms. If it is mounted on the end of the film bag, forming the closure thereof, it may be rotationally symmetrical and adapted to the size of the end of the film bag. The inherently rigid flange may be provided with a guide channel into which the dispensing nozzle is introduced and in which the dispensing nozzle is located when the container is in place. It may be expedient to provide the guide channel with a press fit which surrounds the dispensing nozzle. The press fit may be a part of the guide channel which consists of a smooth inner wall having an internal diameter which differs only slightly from the external diameter of the dispensing nozzle. In another embodiment, a number of bulges may be provided on the inner wall of part of the guide channel. The bulges may for example be three axially extending symmetrically arranged and elongate bulges. In addition, a plurality of bulges arranged at an axial spacing from one another and extending in the azimuthal direction may be provided, which for example form two rings, or which consist of a number of ring sections. In addition the bulges may be spiral in shape; they may consist of a number of spiral sections distributed over the inner wall of the guide channel or of one spiral section the length of which is greater than the circumference of the guide channel. Such a press fit enables the container to be fitted onto the dispensing nozzle and provides a sufficiently firm seat for the inherently rigid flange on the dispensing nozzle. In addition, the container can be pulled off the dispensing nozzle after emptying without damaging the dispensing nozzle.

The inherently rigid flange consists of rubber, metal or plastics, preferably a thermoplastic plastics material. It may be expedient to make the inherently rigid flange from the same plastics from which the film bag or the inside of the film bag is made.

The weld seam at one or both ends of the film bag may be U-, V- or T-shaped; it runs substantially at right angles to the axis of the bag. It may extend partly in the direction of the axis of the bag, thus promoting the defined deformation of the film bag as the fluid is withdrawn.

A sealing point may be provided inside or at one end of the guide channel. The sealing point may consist of a ring which is located in a groove formed on the inner wall of the guide channel. The cross section of the ring may be O-shaped or substantially rectangular. The ring is optionally provided with a sealing lip. The ring may consist of an elastomer, a thermoplastic elastomer or rubber. The sealing point closes off the interior of the container fitted onto the dispensing nozzle against the ambient air in a gas- and fluid-tight manner. It allows the empty container to be pulled off the dispensing nozzle. The sealing point is needed in case the sealing action of the press fit is not sufficient.

The removal point is preferably constructed as a piercing point. A perforatable membrane may be provided at the piercing point, and this membrane is perforated when the container is placed on the dispensing nozzle. The membrane is preferably arranged between the sealing point and the liquid space in the film bag. The perforatable membrane may be provided at one end or inside the guide channel. It is preferably mounted directly on the end of the guide channel or close to this end that faces the liquid space. It may be part of the inherently rigid flange or part of the film bag. If it is part of the inherently rigid flange, it may be produced at the same time as the inherently rigid flange. It may be made of the same plastics as the inherently rigid flange. The perforatable membrane acts as an original seal for the interior of the film bag.

In another embodiment the removal point may be sealed by means of a sealing film which is pulled off before the container is placed on the dispensing nozzle, or is pierced as the container is placed on the dispensing nozzle.

The inherently rigid flange may be in one section or several sections. The multi-sectional flange may preferably be in two sections. The outer section of the flange is tightly connected to the film bag. The outer part contains an opening which is tightly sealed with the inner part. The two parts may be screwed together by means of a thread, or may be joined together by a snap-fit connection or by ultrasonic welding. The one-piece flange is formed analogously to the two-part flange but contains no connecting elements. The inherently rigid flange may be produced at the same time as the press fit, the groove for the sealing point and the perforatable membrane.

The film bag may consist of a tube which has no weld seam extending in the axial direction of the film bag. In addition, it may be made from a film and have one or two weld seams extending in the longitudinal direction. It may be constructed as a flat bag or as a bag with side pleats. A bag with one longitudinally extending weld seam is preferred.

The weld seams on the film bag may be from 0.7 mm to 3 mm wide; their width is selected in accordance with the requirements as to the sealing properties and the durability of the seam. Broad longitudinal seams on the film bag may be bent round after welding so as to abut substantially on the outside of the film bag and so that the film bag is only slightly wider than its width in the non-welded part between the weld seams.

The film bag may consist of a metal or metal alloy foil—preferably of aluminium, gold or copper—or of a plastics film, preferably a thermoplastic. In another embodiment, the film bag may consist of a composite film of plastics and metal. The composite film preferably consists of two or three films joined together. In addition, the film bag may consist of a plastics film which is applied to a layer of metal, glass or ceramics, for example by vapour deposition. The films of plastic or metal are a few microns thick. The thickness of the vapour-deposited layers of metal, glass or ceramics is in the sub-micron range.

The composite film comprising two films may consist of a metal foil and a plastics film which are joined together. The metal foil forms the inside or outside of the composite film. In another embodiment the composite film consists of two different plastics.

The composite film comprising three films preferably consists of two plastics films between which is provided a metal foil. All three films are joined together. Instead of the metal foil there may be a layer of glass or ceramics, for example silicon oxide (SiOₓ) which is vapour-deposited onto a plastics film.

In another embodiment the inner film of the composite film consists of a copolymer, for example a polyethylene copolymer of ethylene-acrylic acid. For the outer plastics film of the composite film a plastics is preferably used, for example polyethylene terephthalate, the melting temperature of which is higher than the melting temperature of the plastics of the inner film. This makes it easier to weld the plastics of the inner film to form a seam when producing the film bag. In the composite film, an adhesion promoting layer may optionally be provided between two films.

The film bag may consist of a plastics film 20 μm to 100 μm thick. It may also consist of a composite film with an inner film of plastics 20 μm to 100 μm thick and an outer film of metal 8 μm to 20 μm thick. It may also consist of a composite film with an inner film of plastics 20 μm to 100 μm thick, a middle film of metal 8 μm to 20 μm thick and an outer film of plastics 10 μm to 40 μm thick.

The weld seams on the film bag and the weld point between the film bag and the inherently rigid flange are produced by known methods such as thermal welding, ultrasonic welding or induction welding for composite films with a metal layer, the weld points preferably being pressed together in the heated state. Methods of this kind are described for example in EP-0 111 131 and EP-0 130 239.

An inherently rigid flange made of rubber or metal may be attached to the film bag by adhesion or optionally by vulcanisation.

The container may be located in an inherently rigid sleeve of metal or plastics, one end of which is detachably or non-detachably connected to the inherently rigid flange, while the other end is optionally closed off by a base. The sleeve may be substantially sealed off all rounds. However, it contains at least one opening or there is a gap at the point of attachment to the flange. In addition, the sleeve may be constructed as an inherently rigid basket with a plurality of openings. The container may be located in an inherently rigid U-shaped bracket instead of the sleeve, the end of each leg of the bracket being attached to the inherently rigid flange and the legs being longer than the film bag. The container located in a sleeve is only attached to the sleeve at the inherently rigid flange. The end sealed with a weld seam or the two ends of the film bag sealed with a weld seam are not attached to the sleeve.

As liquid travels from the container into the dispensing nozzle the film bag collapses flat as a result of the action of external pressure. Air enters the space between the sleeve and the film bag through the opening in the sleeve or through the gap between the sleeve and the inherently rigid flange and thus causes equalisation of pressure. Thus there is no need for a valve in the film bag, and the liquid in the film bag does not come into contact with the air.

The film bag is diffusion-proof for the medicinal fluid and its constituents and for gases. The material for the film bag and optionally the construction of the composite film are selected accordingly. Diffusion-proof for the purposes of the present invention means that there is a loss of liquid (measured with ethanol at ambient temperature) from the container by diffusion of less than 0.6 mg per day, preferably less than 0.4 mg per day, most preferably less than 0.2 mg per day and especially less than 0.1 mg per day.

The inner film or the inside of the film bag is in contact with the liquid introduced therein. This film is made from a material which is not attached by the liquid and which does not have a deleterious effect on the liquid. This film is preferably designed to be weldable.

One of the films or a layer applied by vapour deposition, for example, is the diffusion barrier which prevents the diffusion of the liquid or of its constituents and the diffusion of gases out of or into the film bag. It may be expedient to protect the diffusion barrier from mechanical damage and from tearing when the film is bent by means of another plastics film applied to the diffusion barrier, so as to prevent the diffusion of liquid or gases long-term.

As the film bag is diffusion-proof against gases, the reduced pressure in the film bag caused by the removal of liquid cannot be compensated by the inward diffusion of gas, and the film bag reliably collapses even when fluid is removed from the container very slowly. The liquid can also be removed from the film bag in numerous small amounts, e.g. 200 doses, spread over a fairly long time, e.g. three months.

The container located in a substantially closed sleeve is inaccessible from the outside and cannot be damaged during storage and when placed on the dispensing nozzle. The substantially sealed sleeve or the sleeve constructed as a basket with a plurality of openings or the inherently rigid bracket make it easier to store the container with the thin-walled film bag and to handle it while placing it on the dispensing nozzle and when removing the empty container from the dispensing nozzle.

The dispensing nozzle is, for example, the hollow piston of an atomiser for medicinal fluids. An atomiser of this kind is described in DE-195 36 902.5 and in WO-97/12687 (particularly in FIGS. 6a and 6b therein). The hollow piston of this atomiser is constructed as a dispensing nozzle for the medicinal liquid contained in the container according to the invention. The container is placed on the hollow piston which is preferably mounted along the axis of the atomiser, the end of the hollow piston penetrating into the dispensing nozzle and thus dipping into the medicinal liquid. The sealing point in the inherently rigid flange tightly seals the interior of the container from the outer wall of the hollow piston. The press fit can mechanically secure the container on the hollow piston.

It may be useful to provide a releasable, interlockingly engaging connection between the inherently rigid flange of the container and the dispensing device, e.g. an atomiser, instead of or in addition to the press fit (frictionally engaging connection) between the container and the dispensing nozzle. Such a connection, being a push-in snap-fit connection, may consist of a plurality of snap hooks which are mounted in a connecting member in the dispensing device. When the container is pushed into the dispensing device the snap hooks engage in a recess in the flange, for example in an encircling groove or behind an edge of the inherently rigid flange. The snap-fit lugs are preferably round or chamfered in both directions of movement of the container so that by the application of moderate force an empty container can be removed and a full container can be fitted into the dispensing device.

The container according to the invention is particularly suitable as a replaceable cartridge for inhalable medicament solutions in propellant-free atomisers. The capacity of the container may be from 0.5 ml to 5 ml, preferably from 1 ml to 4 ml and particularly preferably from 1 ml to 3 ml or from 2 ml to 4 ml. These solutions are dispensed batchwise in doses of from 10 microlitres to 5 microlitres, preferably from 15 μl to 20 μl.

The sleeve diameter may be from 10 mm to 30 mm, preferably from 12 mm to 17 mm. The length of the container including the part of the inherently rigid flange protruding from the sleeve may be from 20 mm to 60 mm, preferably from 30 mm to 50 mm.

The formulation examples given below serve to illustrate the present invention without restricting the object of the invention to the particular compounds mentioned by way of example.

### EXAMPLES

Al already mentioned, the compounds of formula 1 may be prepared in known manner. Compounds mentioned by way of example and preferred within the scope of the invention are listed below. Preferred medicament formulations are thus those which contain an active substance 2 and compounds of general formula 1 which are selected from among:

### Example 1

6-hydroxy-8-{1-hydroxy-2-[2-(4-hydroxy-2,6-dimethyl-phenyl)-1,1-dimethyl-ethylamino]-ethyl}-4H-benzo[1,4]oxazin-3-one-methanesulphonate

### Example 2

acid addition salt of 8-{2-[2-(4-fluoro-phenyl)-1,1-dimethyl-ethylamino]-1-hydroxy-ethyl}-6-hydroxy-4H-benzo[1,4]oxazin-3-one

### Example 3

6-hydroxy-8-{1-hydroxy-2-[2-(4-methoxy-phenyl)-1,1-dimethyl-ethylamino]-ethyl}-4H-benzo[1,4]oxazin-3-one-hydrochloride

### Example 4

6-hydroxy-8-{1-hydroxy-2-[2-(ethyl 4-phenoxy-acetate)-1,1-dimethyl-ethylamino]-ethyl}-4H-benzo[1,4]oxazin-3-one-hydrochloride

### Example 5

6-hydroxy-8-{1-hydroxy-2-[2-(4-phenoxy-acetic acid)-1,1-dimethyl-ethylamino]-ethyl}-4H-benzo[1,4]oxazin-3-one-hydrochloride

### Example 6

8-{2-[1,1-dimethyl-2-(2.4.6-trimethylphenyl)-ethylamino]-1-hydroxy-ethyl}-6-hydroxy-4H-benzo[1,4]oxazin-3-one-hydrochloride

### Example 7

6-hydroxy-8-{1-hydroxy-2-[2-(4-hydroxy-phenyl)-1,1-dimethyl-ethylamino]-ethyl}-4H-benzo[1,4]oxazin-3-one-hydrochloride

### Example 8

6-hydroxy-8-{1-hydroxy-2-[2-(4-isopropyl-phenyl)-1,1-dimethyl-ethylamino]-ethyl}-4H-benzo[1,4]oxazin-3-one-hydrochloride

### Example 9

8-{2-[2-(4-ethyl-phenyl)-1,1-dimethyl-ethylamino]-1-hydroxy-ethyl}-6-hydroxy-4H-benzo[1,4]oxazin-3-one-hydrochloride

### Example 10

8-{2-[2-(4-fluoro-3-methyl-phenyl)-1,1-dimethyl-ethylamino]-1-hydroxy-ethyl}-6-hydroxy-4H-benzo[1,4]oxazin-3-one-hydrochloride

### Example 11

8-{2-[2-(4-fluoro-2-methyl-phenyl)-1,1-dimethyl-ethylamino]-1-hydroxy-ethyl}-6-hydroxy-4H-benzo[1,4]oxazin-3-one-hydrochloride

### Example 12

8-{2-[2-(2,4-difluoro-phenyl)-1,1-dimethyl-ethylamino]-1-hydroxy-ethyl}-6-hydroxy-4H-benzo[1,4]oxazin-3-one-hydrochloride

### Example 13

8-{2-[2-(3,5-difluoro-phenyl)-1,1-dimethyl-ethylamino]-1-hydroxy-ethyl}-6-hydroxy-4H-benzo[1,4]oxazin-3-one-hydrochloride

### Example 14

8-{2-[2-(4-ethoxy-phenyl)-1,1-dimethyl-ethylamino]-1-hydroxy-ethyl}-6-hydroxy-4H-benzo[1,4]oxazin-3-one-hydrochloride

### Example 15

8-{2-[2-(3,5-dimethyl-phenyl)-1,1-dimethyl-ethylamino]-1-hydroxy-ethyl}-6-hydroxy-4H-benzo[1,4]oxazin-3-one-hydrochloride

### Example 16

acid addition salt of 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

### Example 17

8-{2-[2-(3,4-difluoro-phenyl)-1,1-dimethyl-ethylamino]-1-hydroxy-ethyl}-6-hydroxy-4H-benzo[1,4]oxazin-3-one-trifluoroacetate

### Example 18

8-{2-[2-(2-chloro-4-fluoro-phenyl)-1,1-dimethyl-ethylamino]-1-hydroxy-ethyl}-6-hydroxy-4H-benzo[1,4]oxazin-3-one-trifluoroacetate

### Example 19

acid addition salt of 8-{2-[2-(4-chloro-phenyl)-1,1-dimethyl-ethylamino]-1-hydroxy-ethyl}-6-hydroxy-4H-benzo[1,4]oxazin-3-one

### Example 20

acid addition salt of 8-{2-[2-(4-bromo-phenyl)-1,1-dimethyl-ethylamino]-1-hydroxy-ethyl}-6-hydroxy-4H-benzo[1,4]oxazin-3-one

### Example 21

acid addition salt of 8-{2-[2-(3-methyl-phenyl)-1,1-dimethyl-ethylamino]-1-hydroxy-ethyl}-6-hydroxy-4H-benzo[1,4]oxazin-3-one;

### Example 22

acid addition salt of 8-{2-[2-(4-fluoro-3-methoxy-phenyl)-1,1-dimethyl-ethylamino]-1-hydroxy-ethyl}-6-hydroxy-4H-benzo[1,4]oxazin-3-one;

### Example 23

acid addition salt of 8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;

### Example 24

acid addition salt of 8-{2-[2-(4-chloro-2-methyl-phenyl)-1,1-dimethyl-ethylamino]-1-hydroxy-ethyl}-6-hydroxy-4H-benzo[1,4]oxazin-3-one;

### Example 25

acid addition salt of 8-{2-[2-(4-chloro-3-fluoro-phenyl)-1,1-dimethyl-ethylamino]-1-hydroxy-ethyl}-6-hydroxy-4H-benzo[1,4]oxazin-3-one;

### Example 26

acid addition salt of 8-{2-[2-(4-chloro-2-fluoro-phenyl)-1,1-dimethyl-ethylamino]-1-hydroxy-ethyl}-6-hydroxy-4H-benzo[1,4]oxazin-3-one;

### Example 27

acid addition salt of 8-{2-[2-(3-chloro-4-fluoro-phenyl)-1,1-dimethyl-ethylamino]-1-hydroxy-ethyl}-6-hydroxy-4H-benzo[1,4]oxazin-3-one;

### Example 28

acid addition salt of 8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;

### Example 29

acid addition salt of 8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;

### Example 30

acid addition salt of 8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;

### Example 31

acid addition salt of 8-{2-[2-(3,5-dichloro-phenyl)-1,1-dimethyl-ethylamino]-1-hydroxy-ethyl}-6-hydroxy-4H-benzo[1,4]oxazin-3-one;

### Example 32

acid addition salt of 8-{2-[2-(4-chloro-3-methyl-phenyl)-1,1-dimethyl-ethylamino]-1-hydroxy-ethyl}-6-hydroxy-4H-benzo[1,4]oxazin-3-one;

### Example 33

acid addition salt of 8-{2-[2-(3,4,5-trifluoro-phenyl)-1,1-dimethyl-ethylamino]-1-hydroxy-ethyl}-6-hydroxy-4H-benzo[1,4]oxazin-3-one;

### Example 34

acid addition salt of 8-{2-[2-(3,4-dichloro-phenyl)-1,1-dimethyl-ethylamino]-1-hydroxy-ethyl}-6-hydroxy-4H-benzo[1,4]oxazin-3-one.
   optionally in the form of an acid addition salt with an acid HX, wherein X⁻ may have one of the meanings given above, and optionally in the form of the tautomers, enantiomers, mixtures of the enantiomers, racemates, solvates or hydrates thereof.

The following Table shows a compilation of formulation examples according to the invention. The abbreviation EDTA denotes disodium edetate-dihydrate and the abbreviation BHT denotes butylhydroxytoluene.

The active substances 1 and 2 specified are optionally used in the form of the salts and/or hydrates thereof, but here they are given in relation to the mass of the free base. Compound 1 is used in the Examples that follow in the form of the hydrochloride, hydrotetrafluoroacetate or hydromethanesulphonate.

A) The following Table shows examples of formulations according to the invention of the R-enantiomer of the compound of Example 1 and the active substance 2. 100 ml of medicament preparation contain:

| | 1′ | | ethanol/ | α- | | | |
|---|---|---|---|---|---|---|---|
| Exam- | (base) | 2 | water | tocopherol | BHT | EDTA | pH |
| ple | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

B) The following Table shows examples of formulations according to the invention of the R-enantiomer of the compound of Example 3 and the active substance 2. 100 ml of medicament preparation contain:

| | 1′ | | ethanol/ | α- | | | |
|---|---|---|---|---|---|---|---|
| Exam- | (base) | 2 | water | tocopherol | BHT | EDTA | pH |
| ple | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

C) The following Table shows examples of formulations according to the invention of the R-enantiomer of the compound of Example 7 and the active substance 2. 100 ml of medicament preparation contain:

| | 1′ | | ethanol/ | α- | | | |
|---|---|---|---|---|---|---|---|
| Exam- | (base) | 2 | water | tocopherol | BHT | EDTA | pH |
| ple | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

D) The following Table shows examples of formulations according to the invention of the R-enantiomer of the compound of Example 9 and the active substance 2. 100 ml of medicament preparation contain:

| | 1′ | | ethanol/ | α-toco- | | | |
|---|---|---|---|---|---|---|---|
| | (base) | 2 | water | pherol | BHT | EDTA | pH |
| Example | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

E) The following Table shows examples of formulations according to the invention of the R-enantiomer of the compound of Example 14 and the active substance 2. 100 ml of medicament preparation contain:

| | 1′ | | ethanol/ | α-toco- | | | |
|---|---|---|---|---|---|---|---|
| | (base) | 2 | water | pherol | BHT | EDTA | pH |
| Example | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

F) The following Table shows examples of formulations according to the invention of the R-enantiomer of the compound of Example 17 and the active substance 2. 100 ml of medicament preparation contain:

| | 1′ | | ethanol/ | α-toco- | | | |
|---|---|---|---|---|---|---|---|
| | (base) | 2 | water | pherol | BHT | EDTA | pH |
| Example | (mg) | (mg) | (% V/V) | (mg) | (mg) | (mg) | (HCl) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 3.0 |
| 5 | 45 | 400 | 70 | — | 100 | 1 | 3.0 |
| 6 | 45 | 800 | 80 | — | — | 0.5 | 3.0 |
| 7 | 45 | 250 | 80 | — | — | 1 | 5.0 |
| 8 | 100 | 1200 | 80 | — | 100 | — | 4.0 |
| 9 | 45 | 1200 | 80 | 50 | — | 0.5 | 3.5 |
| 10 | 100 | 1000 | 90 | 50 | — | — | 3.0 |
| 11 | 100 | 2000 | 90 | — | 100 | — | 3.0 |
| 12 | 9 | 2500 | 90 | — | — | 0.5 | 4.0 |
| 13 | 45 | 2000 | 90 | 50 | — | — | 5.0 |
| 14 | 45 | 2000 | 90 | — | 100 | — | 4.5 |
| 15 | 45 | 2500 | 90 | — | — | 1 | 5.0 |
| 16 | 45 | 2000 | 90 | 50 | — | 0.5 | 4.0 |
| 17 | 45 | 2000 | 90 | — | 100 | 1 | 5.0 |
| 18 | 100 | 2000 | 95 | 50 | 100 | — | 4.0 |
| 19 | 45 | 4000 | 95 | — | 100 | 0.5 | 5.0 |
| 20 | 9 | 2500 | 95 | 50 | — | — | 4.5 |

G) The following Table shows examples of formulations according to the invention of the R-enantiomer of the compound of Example 1 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

H) The following Table shows examples of formulations according to the invention of the R-enantiomer of the compound of Example 3 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

I) The following Table shows examples of formulations according to the invention of the R-enantiomer of the compound of Example 7 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

J) The following Table shows examples of formulations according to the invention of the R-enantiomer of the compound of Example 9 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

K) The following Table shows examples of formulations according to the invention of the R-enantiomer of the compound of Example 14 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

L) The following Table shows examples of formulations according to the invention of the R-enantiomer of the compound of Example 17 and the active substance 2. 100 ml of medicament preparation contain:

| | | | ethanol/ | | | | pH |
|---|---|---|---|---|---|---|---|
| | 1′ | | water | α-toco- | | | (H₃PO₄ |
| | (base) | 2 | (% | pherol | BHT | EDTA | or |
| Example | (mg) | (mg) | V/V) | (mg) | (mg) | (mg) | buffer) |
| 1 | 9 | 400 | 70 | — | 100 | — | 4.5 |
| 2 | 9 | 250 | 70 | 50 | — | — | 5.0 |
| 3 | 45 | 500 | 70 | — | — | 4 | 4.0 |
| 4 | 100 | 400 | 70 | 50 | — | 0.5 | 5.5 |
| 5 | 45 | 400 | 70 | — | 100 | 1 | 5.0 |
| 6 | 9 | 600 | 70 | — | 50 | 2 | 6.0 |
| 7 | 45 | 800 | 80 | — | — | 1 | 4.5 |
| 8 | 45 | 250 | 80 | — | — | 2 | 5.0 |
| 9 | 100 | 1200 | 80 | — | 100 | — | 4.0 |
| 10 | 45 | 1200 | 80 | 50 | — | 1 | 4.5 |
| 11 | 9 | 1600 | 80 | — | 100 | 2 | 5.5 |
| 12 | 45 | 1600 | 80 | 50 | — | 3 | 6.0 |
| 13 | 100 | 1000 | 90 | 50 | — | — | 4.5 |
| 14 | 100 | 2000 | 90 | — | 100 | — | 5.0 |
| 15 | 9 | 2500 | 90 | — | — | 0.5 | 4.0 |
| 16 | 45 | 2000 | 90 | 50 | — | — | 5.0 |
| 17 | 45 | 2000 | 90 | — | 100 | — | 4.5 |
| 18 | 45 | 2500 | 90 | — | — | 1 | 5.0 |
| 19 | 45 | 2000 | 90 | 50 | — | 0.5 | 6.0 |
| 20 | 45 | 2000 | 90 | — | 100 | 1 | 5.0 |
| 21 | 100 | 3500 | 90 | 50 | — | 1 | 5.5 |
| 22 | 45 | 3500 | 90 | — | 50 | 0.5 | 5.5 |
| 23 | 100 | 2000 | 95 | 50 | 100 | — | 5.0 |
| 24 | 45 | 4000 | 95 | — | 100 | 0.5 | 5.5 |
| 25 | 9 | 2500 | 95 | 50 | — | — | 4.5 |

M) The following Table shows examples of formulations according to the invention of the R-enantiomer of the compound of Example 1 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

N) The following Table shows examples of formulations according to the invention of the R-enantiomer of the compound of Example 3 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

O) The following Table shows examples of formulations according to the invention of the R-enantiomer of the compound of Example 7 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

P) The following Table shows examples of formulations according to the invention of the R-enantiomer of the compound of Example 9 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

Q) The following Table shows examples of formulations according to the invention of the R-enantiomer of the compound of Example 14 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

L) The following Table shows examples of formulations according to the invention of the R-enantiomer of the compound of Example 17 and the active substance 2. 100 ml of medicament preparation contain:

| | | | ethanol/ | | | | pH |
|---|---|---|---|---|---|---|---|
| | 1′ | | water | α-toco- | | | (HCl |
| | (base) | 2 | (% | pherol | BHT | EDTA | or |
| Example | (mg) | (mg) | V/V) | (mg) | (mg) | (mg) | buffer) |
| 1 | 7 | 735 | 70 | — | 100 | — | 4.5 |
| 2 | 7 | 1471 | 70 | 50 | — | — | 5.0 |
| 3 | 30 | 368 | 70 | — | — | 4 | 4.0 |
| 4 | 120 | 735 | 70 | 50 | — | 0.5 | 5.5 |
| 5 | 30 | 1471 | 70 | — | 100 | 1 | 5.0 |
| 6 | 15 | 735 | 70 | — | 50 | 2 | 5.5 |
| 7 | 30 | 1471 | 80 | — | — | 1 | 4.5 |
| 8 | 30 | 735 | 80 | — | — | 2 | 5.0 |
| 9 | 120 | 2942 | 80 | — | 100 | — | 4.0 |
| 10 | 15 | 2942 | 80 | 50 | — | 1 | 4.5 |
| 11 | 7 | 1471 | 80 | — | 100 | 2 | 5.5 |
| 12 | 30 | 2942 | 80 | 50 | — | 3 | 6.0 |
| 13 | 120 | 2942 | 90 | 50 | — | — | 4.5 |
| 14 | 120 | 1471 | 90 | — | 100 | — | 5.0 |
| 15 | 7 | 735 | 90 | — | — | 0.5 | 4.0 |
| 16 | 30 | 735 | 90 | 50 | — | — | 5.0 |
| 17 | 30 | 1471 | 90 | — | 100 | — | 4.5 |
| 18 | 30 | 2942 | 90 | — | 50 | — | 5.0 |
| 19 | 30 | 735 | 90 | 50 | — | 0.5 | 5.5 |
| 20 | 30 | 6000 | 90 | — | 100 | 0.5 | 5.0 |
| 21 | 60 | 2942 | 90 | 50 | — | 1 | 5.5 |
| 22 | 30 | 2942 | 90 | — | 50 | 0.5 | 4.5 |
| 23 | 120 | 2942 | 95 | 50 | 100 | — | 5.0 |
| 24 | 30 | 6000 | 95 | — | 100 | 0.5 | 5.5 |
| 25 | 7 | 1471 | 95 | 50 | — | — | 4.5 |

1. Medicament formulation comprising:
as active substance one or more compounds of general formula 1 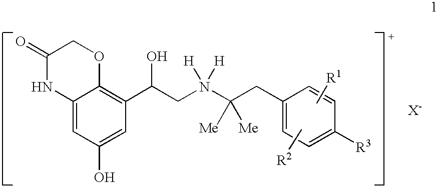
wherein
R¹ denotes hydrogen, C₁₋₄-alkyl, O—C₁₋₄-alkyl or halogen;
R² denotes hydrogen, C₁₋₄-alkyl, O—C₁₋₄-alkyl or halogen;
R³ denotes hydrogen, C₁₋₄-alkyl, O—C₁₋₄-alkyl, halogen, OH, —O—C₁₋₄-alkylene-COOH or O—C₁₋₄-alkylene-COO—C₁₋₄-alkyl;
X⁻ denotes a mono- or polysubstituted negatively charged anion;
an active substance 2 selected from budesonide, beclomethasone, fluticasone and ciclesonide, or a metabolite thereof; and
at least one pharmacologically acceptable acid or a pharmacologically acceptable buffer system.

2. Medicament formulation according to claim 1, wherein said mono- or polysubstituted negatively charged anion is chloride, bromide, iodide, sulphate, phosphate, methanesulphonate, nitrate, maleate, acetate, benzoate, citrate, salicylate, trifluoroacetate, fumarate, tartrate, oxalate, succinate, benzoate or p-toluenesulphonate.

3. Medicament formulation according to claim 1, further comprising:
pharmacologically acceptable excipients; and
ethanol or a mixture of water and ethanol as the solvent.

4. Medicament formulation according to claim 1, wherein one or more said active substances is in the form of the tautomers, enantiomers, mixtures of the enantiomers, racemates, solvates or hydrates thereof.

5. Medicament formulation according to claim 1, wherein
R¹ denotes hydrogen, methyl, ethyl, fluorine or chlorine;
R² denotes hydrogen, methyl, ethyl, fluorine or chlorine;
R³ denotes hydrogen, methyl, ethyl, propyl, OH, methoxy, ethoxy, fluorine, chlorine, bromine, O—CH₂—COOH, O—CH₂—COOmethyl or O—CH₂—COOethyl, —O—CH₂—CH₂COOH, O—CH₂—CH₂COOmethyl or O—CH₂—CH₂COOethyl, —O—CH₂—CH₂—CH₂COOH, O—CH₂—CH₂—CH₂COOmethyl or —O—CH₂—CH₂—CH₂COOethyl;
X⁻ denotes a mono- or polysubstituted negatively charged anion.

6. Medicament formulation according to claim 5, wherein said mono- or polysubstituted negatively charged anion is chloride, bromide, iodide, sulphate, phosphate, methanesulphonate, nitrate, maleate, acetate, benzoate, citrate, salicylate, trifluoroacetate, fumarate, tartrate, oxalate, succinate, benzoate or p-toluenesulphonate.

7. Medicament formulation according to claim 5, wherein one or more said active substances is in the form of the tautomers, enantiomers, mixtures of the enantiomers, racemates, solvates or hydrates thereof.

8. Medicament formulation according to claim 1, wherein
R¹ denotes hydrogen or methyl;
R² denotes hydrogen or methyl;
R³ denotes methyl, OH, methoxy, fluorine, chlorine, bromine, O—CH₂—COOH or —O—CH₂—COOethyl;
X⁻ denotes a mono- or polysubstituted negatively charged anion selected from chloride, bromide, sulphate, methanesulphonate, maleate, acetate, benzoate, citrate, salicylate, trifluoroacetate, fumarate, tartrate and succinate.

9. Medicament formulation according to claim 8, wherein
R¹ denotes hydrogen.

10. Medicament formulation according to claim 8, wherein
R² denotes hydrogen.

11. Medicament formulation according to claim 8, wherein one or more said active substances is in the form of the tautomers, enantiomers, mixtures of the enantiomers, racemates, solvates or hydrates thereof.

12. Medicament formulation according to claim 1, wherein the active substance 2 is budesonide or ciclesonide, or a metabolite thereof.

13. Medicament formulation according to claim 12, wherein said active substance 2 is in the form of the tautomers, enantiomers, mixtures of the enantiomers, racemates, solvates or hydrates thereof.

14. Medicament formulation according to claim 1, wherein the pharmacologically acceptable acid is selected from the inorganic acids hydrochloric acid, phosphoric acid, hydrobromic acid, nitric acid and sulphuric acid or from the organic acids ascorbic acid, citric acid, malic acid, tartaric acid, maleic acid, succinic acid, fumaric acid, acetic acid, formic acid, propionic acid, sorbic acid, benzoic acid, methanesulphonic acid and benzenesulphonic acid.

15. Medicament formulation according to claim 1, wherein the pharmacologically acceptable buffer system is a citrate buffer, an acetate buffer or a phosphate buffer.

16. Medicament formulation according to claim 1, wherein the pH of said formulation is 2.0 to 6.5.

17. Medicament formulation according to claim 1, wherein the content of 1′ and 2 independently of one another is about 0.1 to 6000 mg per 100 ml solution in each case.

18. Medicament formulation according to claim 1, wherein said formulation comprises a complexing agent as a further pharmacologically acceptable excipient.

19. Medicament formulation according to claim 18, wherein the content of said complexing agent is 0.1 to 50 mg per 100 ml solution.

20. Medicament formulation according to claim 1, wherein said formulation comprises an antioxidant as a further pharmacologically acceptable excipient.

21. Medicament formulation according to claim 1, wherein said formulation comprises as a further pharmacologically acceptable excipient an antioxidant selected from ascorbic acid, propylgallate, butylhydroxyanisol, butylhydroxytoluene, tert-butylhydroxyquinone, tris(2,4-di-tert-butylphenyl)phosphite and tetrakis[methylene(3,5-di-tert-butylhydroxyhydrocinnamate)]methane, tocopherol, naringenin and resveratrol.

22. Medicament formulation according to claim 1, wherein said formulation comprises a mixture of water and ethanol as solvent.

23. Medicament formulation according to claim 1, wherein said formulation comprises benzylalcohol, γ-butyrolactone or diethyleneglycol monoethylether as co-solvent.

24. Medicament formulation according to claim 22, wherein said formulation comprises as solvent a mixture of water and ethanol in which the percentage amount of ethanol by volume is in the range between 30 and 99% ethanol.

25. Medicament formulation comprising:
as active substance a free base of formula 1′ 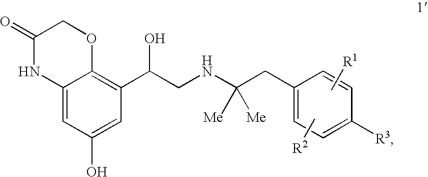
wherein the groups R¹, R² and R³ may have the meanings given in claim 1;
an active substance 2 selected from budesonide, beclomethasone, fluticasone and ciclesonide, or a metabolite thereof; and
at least one pharmacologically acceptable acid.

26. Medicament formulation according to claim 25, further comprising:
pharmacologically acceptable excipients; and
ethanol or a mixture of water and ethanol as the solvent.

27. Medicament formulation according to claim 25, wherein one or more said active substances is in the form of the tautomers, enantiomers, mixtures of the enantiomers, racemates, solvates or hydrates thereof.

28. Medicament formulation according to claim 27, wherein the active substance 2 is budesonide or ciclesonide, or a metabolite thereof.

29. Medicament formulation according to claim 28, wherein one or more of said active substances is in the form of the tautomers, enantiomers, mixtures of the enantiomers, racemates, solvates or hydrates thereof.

30. A method of treating respiratory complaints, said method comprising administering to a patient in need thereof a therapeutically effective amount of a medicament formulation according to claim 1.

31. Inhalation kit consisting of a medicament formulation according to claim 1 and an inhaler suitable for nebulising said medicament formulation.

32. Inhalation kit according to claim 31, wherein the inhaler is a Respimat®.
